(19) United States
(12) Patent Application Publication
(10) Pub. No.: US 2015/0313486 A1
(43) Pub. Date: Nov. 5, 2015

(54) DETERMINING PULSE WAVE TRANSIT TIME FROM PPG AND ECG/EKG SIGNALS

(71) Applicant: Xerox Corporation, Norwalk, CT (US)

(72) Inventors: Lalit Keshav MESTHA, Fairport, NY (US); Survi KYAL, Rochester, NY (US)

(21) Appl. No.: 14/596,344

(22) Filed: Jan. 14, 2015

Related U.S. Application Data
(63) Continuation-in-part of application No. 14/268,656, filed on May 2, 2014.

(51) Int. Cl. A61B 5/024; A61B 5/00; A61B 5/0285; A61B 5/02; A61B 5/0456; A61B 5/021

(52) U.S. Cl. CPC A61B 5/02427; A61B 5/0456; A61B 5/02125; A61B 5/0285; A61B 5/02007; A61B 5/7225; A61B 5/7289

(57) ABSTRACT

What is disclosed is a system and method for determining a pulse transit time for a subject. In one embodiment, the system comprises a handheld wireless cellular device configured with a sensor to acquire an electrocardiogram (ECG) signal from the subject and a photodetector generating a time-series signal in response to continuously sensing a reflection of source light off a region of exposed skin of the subject where a photoplethysmographic (PPG) signal can be detected by the photodetector. In one embodiment, the time-series signal is a PPG signal. In another embodiment, the time-series signal is processed to extract a PPG signal. A temporally overlapping segment of the PPG and ECG signals is analyzed to obtain a pulse transit time between a reference point on the PPG signal and a reference point on the ECG signal. The pulse transit time is used to assess pathologic conditions such as peripheral vascular disease.

### CROSS REFERENCE TO RELATED APPLICATIONS

This patent application is a continuation-in-part of commonly owned and co-pending U.S. patent application Ser. No. 14/268,656, entitled: “Determining Arterial Pulse Wave Transit Time From VPG And ECG/EKG Signals”, by Mestha et al. which is incorporated herein in its entirety by reference.

### TECHNICAL FIELD

The present invention is directed to systems and methods for determining pulse transit time for a subject using a sensor to acquire an electrocardiogram (ECG) signal and a photodetector to obtain a photoplethysmographic (PPG) signal by continuously sensing light reflected off a region of exposed skin where a PPG signal can be detected by that photodetector.

### BACKGROUND

The ability to capture physiological signals is highly desirable in the healthcare industry. One important physiological signal is pulse transit time (PTT). PTT correlates well with blood pressure and can provide healthcare professionals with vital information relating to blood velocity and blood vessel dilation over time. Localized PTT can be used to assess blood vessel blockage between two points and thus can be used as an indirect marker for assessing peripheral vascular disease.

### BRIEF SUMMARY

What is disclosed is a system and method for determining a pulse transit time for a subject. In one embodiment, a handheld wireless cellular device is configured with a sensor to acquire an electrocardiogram (ECG) signal from the subject and a photodetector generating a time-series signal in response to continuously sensing a reflection of source light off a region of exposed skin of the subject where a photoplethysmographic (PPG) signal can be detected by that photodetector. The acquired ECG and PPG signals are received. Thereafter, a temporally overlapping segment of the PPG and ECG signals is then analyzed to obtain a pulse transit time between a reference point on the PPG signal and a reference point on the ECG signal. The pulse transit time can be used to assess pathologic conditions such as peripheral vascular disease. Advantages of the above-described system and method will become apparent from the following detailed description and accompanying drawings.

### BRIEF DESCRIPTION OF THE DRAWINGS

The foregoing and other features and advantages of the subject matter disclosed herein will be made apparent from the following detailed description taken in conjunction with the accompanying drawings, in which:

FIG. 1 shows a handheld wireless cellular device actively capturing PPG and ECG signals from a subject;

FIG. 2 shows a portion of an ECG signal of a normal sinus rhythm obtained by the sensor of FIG. 1;

FIG. 3 is a flow diagram which illustrates one example embodiment of the present method for determining a pulse transit time for a subject as shown and described with respect to the embodiments of FIGS. 1 and 4; and

FIG. 4 illustrates a block diagram of one example signal processing system for performing various aspects of the teachings hereof.

### DETAILED DESCRIPTION

What is disclosed is a system and method for determining a pulse transit time for a subject.

### Non-Limiting Definitions

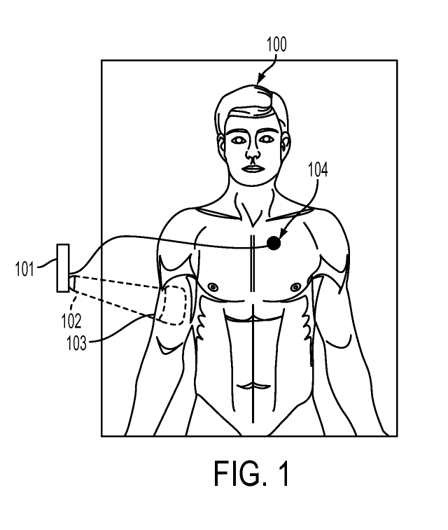

A “subject” refers to a living being. One example subject 100 is shown in FIG. 1. Although the term “person” or “patient” may be used throughout this disclosure, it should be appreciated that the subject may be something other than a human such as, for example, a primate. Therefore, the use of such terms is not to be viewed as limiting the scope of the appended claims strictly to humans.

“Plethysmography” is the study of signals containing relative blood volume changes in vessels which are close to the skin surface.

A “photoplethysmographic signal”, or simply “PPG signal” is a signal obtained using a photodetector.

A “photodetector” is a device which can continuously measure light intensities and generates, as output, a time-series signal. FIG. 1 shows an example handheld wireless cellular device 101 configured with a photodetector 102 to continuously measure light intensities off a surface of a region of exposed skin 104. Photodetector 102 provides the continuous time-series signal to a processor which, in one embodiment, is internal to the wireless cellular device 101.

A “time-series signal” is a signal which contains the sum total of relative blood volume changes in the blood vessels close to the skin surface in the region of exposed skin being sensed. The time-series signal may be filtered with a cutoff frequency defined as a function of a frequency of the subject's cardiac pulse. Filtered time-series signal segments can be upsampled to a pre-selected sampling frequency to increase a total number of data points in order to enhance an accuracy of peak-to-peak pulse point detection. Upsampling may involve, for example, an interpolation technique using a cubic spline function and a pre-selected sampling frequency. The upsampled signals can be smoothed using any of a variety of smoothing techniques which are well established in the arts. In one embodiment, the time-series signal comprises a PPG signal. In another embodiment, the PPG signal is extracted from the time-series signal.

“Extracting a PPG signal” from the time-series signal can be effectuated by performing signal separation on the time-series signals. The obtained signal is converted to a zero-mean unit variance. One method for determining PTT is further disclosed in: “System And Method For Determining Arterial Pulse Wave Transit Time”, U.S. patent application Ser. No. 14/204,397, by Mestha et al. and in “Determining Arterial Pulse Transit Time From Time-Series Signals Obtained At Proximal And Distal Arterial Sites”, U.S. patent application Ser. No. 14/515,618, by Mestha et al., both of which are incorporated herein in their entirety by reference. A filtering step can be performed to improve peak detection accuracy. Additional corrections may be necessary based on estimating the average of the amplitudes obtained from previous peaks. The extracted PPG signal can be filtered using any of: an FFT-based phase preservation filter, a zero-phase digital filter, a linear time invariant (LTI) filter, a linear time varying (LTV) filter, a finite impulse response (FIR) filter, an infinite impulse response (IIR) filter, or a non-linear filter such as a median filter.

A “sensor”, as used herein, is a device which senses the electrical activity of a beating heart. FIG. 1 shows sensor 104 on a skin surface of a chest area of the subject 100. Sensor 104 captures electrocardiogram signals and communicates those signals to the wireless cellular device 101.

An “electrocardiogram (ECG) signal” (alternatively, EKG, from the Greek “kardia”, meaning heart) are well understood by cardiac specialists. For a further discussion of ECG/EKG signals, the reader is directed to the introductory text: “EKGs for the Nurse Practitioner and Physician Assistant”, Springer Publishing Co. (2013), ISBN-13: 978-0826199560.

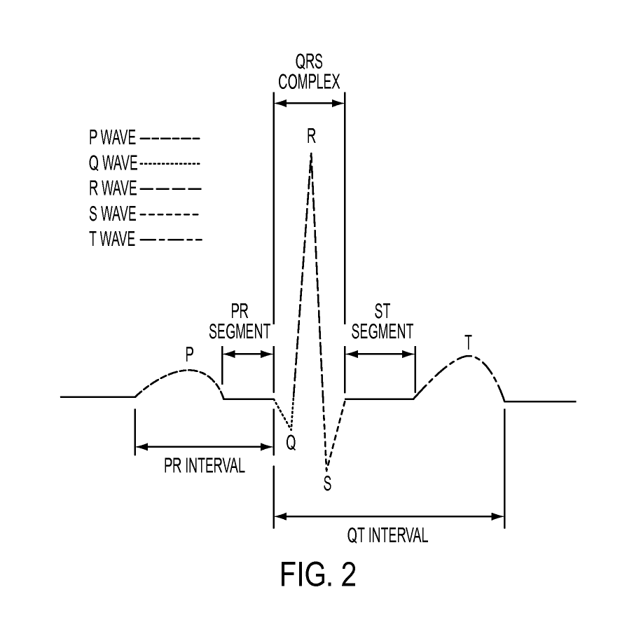

A “reference point on the ECG signal” can be any characteristic point on the ECG signal of FIG. 2 which shows a portion of an ECG signal of a normal sinus rhythm obtained by the sensor 104 of FIG. 1. In one embodiment, the reference point on the ECG signal is a peak point of the R wave.

A “reference point on the PPG signal” can be any characteristic point on the PPG signal. In various embodiments hereof, the reference point on the PPG signal is any of: a maximum or a minimum point on the PPG signal, an average point between a maximum and a minimum on the PPG signal, a maximum of a first derivative of the PPG signal, or a maximum of a second derivative of the PPG signal. A pulse transit time is determined between the reference point on the PPG signal and the reference point on the ECG signal.

“Pulse transit time (PTT)” is the time it takes an arterial pulse pressure wave to travel between two points. An arterial pulse pressure wave is generated when the left ventricle of the heart contracts and pushes a volume of blood out the ascending aorta into the systemic arteries. The repeated push of this blood volume generates a pulsating wave. PTT can be used to determine blood pressure, blood vessel dilation over time, blood vessel blockage, and blood flow velocity. Furthermore, PTT can be used as an indirect marker for assessing the occurrence of cardiac arrhythmia, cardiac stress, heart disease, and peripheral vascular disease. PTT can be determined by averaging an instantaneous PTT over a user defined number of cardiac cycles. In one embodiment, the instantaneous PTT is:

PTT=^{d}/_{η}_{_{HR}}

where η_{HR} is the frequency of the subject's cardiac pulse (in beats per minute) and d is the phase difference (in radians per second) between two reference points on the ECG and PPG signals.

A “phase difference” is obtained as follows:

dφ=φ_{PPG}(t)−φ_{ECG}(t)

where φ_{PPG} is the phase of the PPG signal at time t and φ_{ECG} and is the phase of the

ECG signal at time t. Methods for determining a phase of a signal at a given point in time are well understood. The reader is directed to “Handbook of Formulas and Tables for Signal Processing”, CRC Press, 1^{st} Ed. (1998), ISBN-13: 978-0849385797, which is incorporated herein in its entirety by reference. In certain applications, phase unwrapping may be required for one or both of the φ_{PPG}(t) and φ_{ECG}(t) signals.

“Receiving signals” is intended to be widely construed and includes: retrieving, capturing, acquiring, or otherwise obtaining signals for processing in accordance with the methods disclosed herein. Signals can be retrieved from a memory or storage device, retrieved from a media such as a CDROM or DVD, obtained from a remote device over a network, or downloaded from a web-based system or application which makes such signals available.

It should be appreciated that the steps of “determining”, “analyzing”, “obtaining”, “receiving”, “processing”, “detrending”, “filtering”, “performing” and the like, as used herein, include the application of various signal processing and mathematical operations applied to data and signals, according to any specific context or for any specific purpose. It should be appreciated that such steps may be facilitated or otherwise effectuated by a microprocessor executing machine readable program instructions retrieved from a memory or storage device.

### Example Flow Diagram

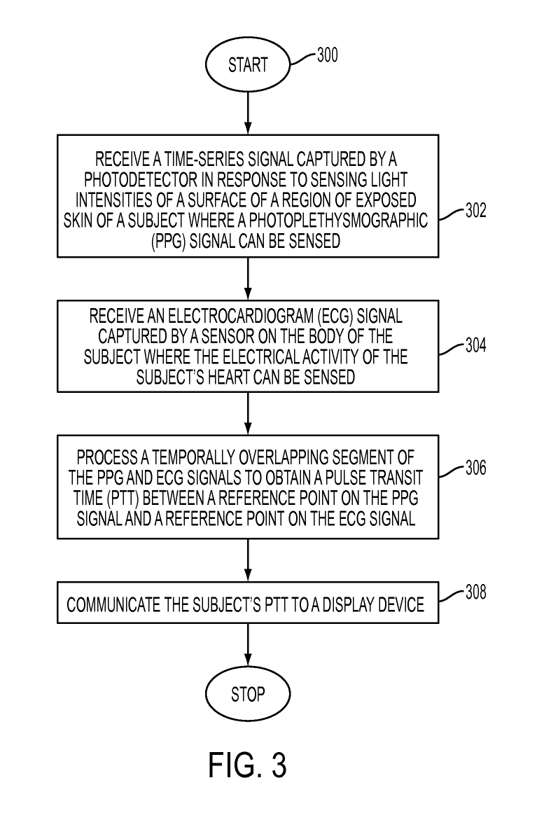

Reference is now being made to the flow diagram of FIG. 3 which illustrates one example embodiment of the present method for determining a pulse transit time for a subject. Flow processing begins at step 300 and immediately proceeds to step 302.

At step 302, receive a time-series signal captured by at least one photodetector in response to sensing light intensities off a surface of a region of exposed skin of a subject where a photoplethysmographic (PPG) signal can be sensed.

At step 304, receive an electrocardiogram (ECG) signal captured by a sensor placed on the subject's body where the electrical activity of the subject's heart can be sensed.

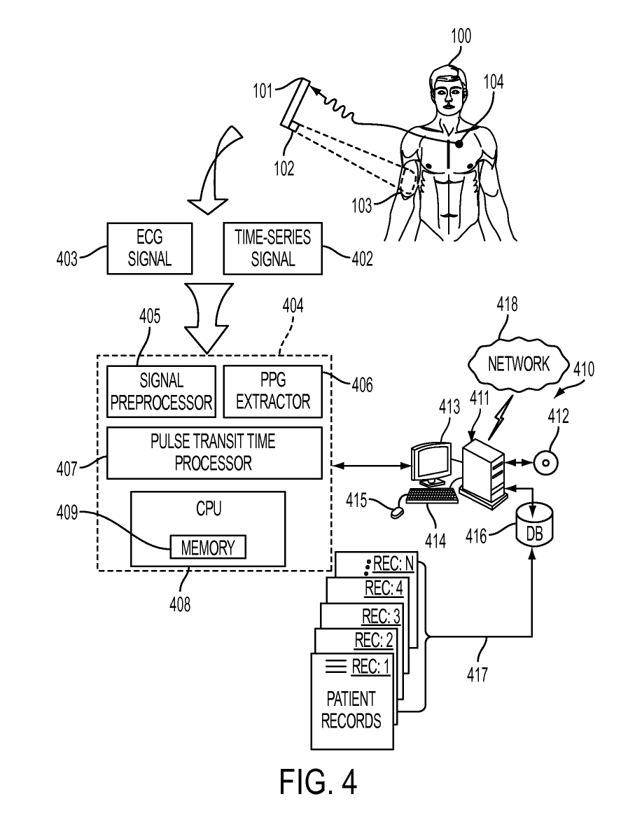

At step 306, process a temporally overlapping segment of the PPG and ECG signals to obtain a pulse transit time (PTT) between a reference point on the PPG signal and a reference point on the ECG signal. A user may use the workstation or the smartphone of FIG. 4 to select the two reference points.

At step 308, communicate the subject's PTT to a display device. One example display device is shown in conjunction with the workstation of FIG. 4. The display device may be the display of the smartphone. In this embodiment, further processing stops.

It should be appreciated that the flow diagrams depicted herein are illustrative. One or more of the operations may be performed in a differing order. Other operations may be added, modified, enhanced, or consolidated. Variations thereof are intended to fall within the scope of the appended claims.

### Example Networked System

Reference is now being made to FIG. 4 which illustrates a block diagram of one example signal processing system for performing various aspects of the teachings hereof for determining a pulse transit time for a subject.

Smartphone 101 receives a time-series signal from a photodetector 402 and an ECG signal from a sensor 104 physically connected to the smartphone. In another embodiment, the sensor 104 is in communication with the smartphone 101 via a wireless protocol. In another embodiment, the smartphone itself is the ECG sensor and it obtains ECG signals by the smartphone being placed in physical contact with a surface of the subject's skin. Both the photodetector 102 and the sensor 104 may be configurable by a software application executed by a processor internal to the smartphone. Such an application displays an icon widget in the form of a button which, when pressed by a user touching the smartphone's touchscreen display, activates both the photodetector and the sensor to begin capture of the time-series signal 402 and the ECG signal 403, respectively. Touching the button to switch to the OFF position stops signal acquisition and initiates signal processing.

In the embodiment of FIG. 4, the signal processing system 404 comprises a Signal Preprocessor Module 405 which detrends and filters the time-series signal 402, as needed. PPG Extractor 406 receives the detrended and filtered time-series signal from Module 405 and proceeds to extract the PPG signal therefrom using, for example, a cICA method. An option to bypass Module 405 may be made by a user by touching an option on a menu displayed on a screen of the smartphone device. A user may further select various displayed menu options to, for instance, set one or more threshold levels for signal acquisition, reject signals, and select reference points on the PPG and ECG signals. Various adjustments can also be dynamically made by a user of the smartphone to the acquired signals such as amplitude, duration, peak detection, and the like. PTT Processor 407 receives the extracted PPG signal from Module 406 and the ECG signal 403 and determines the pulse transit time between two reference points. Central Processor (CPU) 408 retrieves machine readable program instructions from Memory 409. The CPU and Memory, alone or in conjunction with other processors, modules, and memory, may be configured to assist or otherwise facilitate the functionality of any of the units of system 404.

Signal Processing System 404 is shown having been placed in communication with a workstation, generally at 410. A computer case 411 houses various components such as a motherboard with a processor and memory, a network card, a video card, a hard drive capable of reading/writing to machine readable media 412 such as a floppy disk, optical disk, CD-ROM, DVD, magnetic tape, and the like, and other software and hardware needed to perform the functionality of a computer workstation. The workstation further includes a display device 413, such as a CRT, LCD, or touchscreen device, for displaying information and various user-selectable menu options, PPG signals, ECG signals, computed values, patient medical information, results, and the like. A user can view any of that information and make a selection from menu options displayed thereon or directly from the screen of the smartphone 102. Keyboard 414 and mouse 415 effectuate a user input or user selection. The workstation implements a database in storage device 416 wherein records are stored, manipulated, and retrieved in response to a query. Such records, in various embodiments, take the form of patient medical history (collectively at 417) stored in association with information identifying the patient. Although the database is shown as an external device, the database may be internal to the workstation mounted, for example, on a hard disk mounted internally to computer case 411.

The workstation 410 has an operating system and other specialized software configured to display alphanumeric values, menus, scroll bars, dials, slideable bars, pull-down options, selectable buttons, and the like, for entering, selecting, modifying, and accepting information needed for processing image frames, time-series signals, PPG signals, and ECG signals, in accordance with the teachings hereof. The workstation is further enabled to display the various signals for a user to review and to select references points on those signals. In other embodiments, a user or technician uses the user interface of the workstation (or the interface of the smartphone) to set parameters, view and analyze signals, and the like. User selections may be stored/retrieved in any of the storages devices 412 and 416. Default settings and initial parameter values can be stored/retrieved from any of the storage devices.

Although shown as a desktop computer, it should be appreciated that the workstation 410 can be a laptop, mainframe, or a special purpose computer such as an ASIC, circuit, or the like. The workstation of FIG. 4 is illustrative and may include other functionality known in the arts. Any of the components of the workstation may be placed in communication with the Signal Processing System 404 or any devices in communication therewith. Any of the modules and processing units of system 404 can be placed in communication with the database 416 and/or computer readable media 412 and may store/retrieve therefrom data, variables, records, parameters, functions, and/or machine readable/executable program instructions, as needed to perform their intended functions. Each of the modules of the system 404 may be placed in communication with one or more remote devices over network 418.

It should be appreciated that some or all of the functionality performed by any of the modules or processing units of system 404 can be performed, in whole or in part, by the workstation 410 placed in communication with the smartphone 101 over network 418. It should also be understood that any of the functionality performed by the system 404 and/or the workstation 410 may be performed, in whole or in part, by the smartphone 101. The embodiment shown should not be viewed as limiting the scope of the appended claims strictly to that configuration. Various modules may designate one or more components which may, in turn, comprise software and/or hardware designed to perform the intended function.

### Various Embodiments

The above-disclosed and other features and functions, or alternatives thereof, may be desirably combined into other different systems or applications. The teachings hereof can be implemented in hardware or software using any known or later developed systems, structures, devices, and/or software by those skilled in the applicable art without undue experimentation from the functional description provided herein with a general knowledge of the relevant arts. One or more aspects of the methods described herein are intended to be incorporated in an article of manufacture which may be shipped, sold, leased, or otherwise provided separately either alone or as part of a product suite or a service. Presently unforeseen or unanticipated alternatives, modifications, variations, or improvements may become apparent and/or subsequently made by those skilled in this art which are also intended to be encompassed by the following claims. The teachings of any publications referenced herein are each hereby incorporated by reference in their entirety.

What is claimed is:

1. A method for determining pulse wave transit time for a subject, comprising:
receiving a time-series signal generated by a photodetector sensing an intensity of light reflected off a region of exposed skin of a subject where a photoplethysmographic (PPG) signal can be detected, said time-series signal comprising said PPG signal;
receiving an electrocardiogram (ECG) signal obtained by a sensor placed on said subject's body where electrical activity of said subject's heart can be sensed; and
processing a temporally overlapping segment of said PPG and ECG signals to determine a pulse transit time between a reference point on said PPG signal and a reference point on said ECG signal.

2. The method of claim 1, wherein, in advance of processing said temporally overlapping segment of said PPG and ECG signals, further comprising compensating for motion in said PPG signal induced by a movement of said subject.

3. The method of claim 1, wherein, in advance of processing said temporally overlapping segment of said PPG and ECG signals, further comprising any of:
detrending said time-series signal to remove non-stationary components;
filtering said time-series signal with a cutoff frequency defined as a function of a frequency of said subject's cardiac pulse; and
performing automatic peak detection on said filtered signal.

4. The method of claim 1, wherein, in advance of processing said temporally overlapping segment of said PPG and ECG signals, further comprising temporally synchronizing said PPG and ECG signals.

5. The method of claim 1, wherein said reference point on said PPG signal is a characteristic point comprising any of: a maximum or a minimum point on said PPG signal, an average point between a maximum and a minimum on said PPG signal, a maximum of a first derivative of said PPG signal, and a maximum of a second derivative of said PPG signal.

6. The method of claim 1, wherein said reference point on said ECG signal is a peak point of a R wave.

7. The method of claim 1, wherein said reference point on said PPG signal and said reference point on said ECG signal are selected by a user.

8. The method of claim 1, wherein determining said pulse transit time (PTT) between said two reference points comprises:
calculating an instantaneous PTT at least comprising:
PTT=^{d}/_{η}_{_{HR}}
where η_{HR} is a frequency of said subject's cardiac pulse in beats/minute and d is a phase difference in radians/second between said two reference points on said temporally synchronous ECG and PPG signals; and
averaging said instantaneous PTT over at least two cardiac cycles.

9. The method of claim 1, wherein said PPG signal is extracted from said time-series signal.

10. The method of claim 1, further comprising determining, from said pulse transit time, any of: blood pressure, blood vessel dilation over time, blood vessel blockage, and blood flow velocity.

11. The method of claim 10, further comprising determining an occurrence of any of:
cardiac arrhythmia, cardiac stress, heart disease, and peripheral vascular disease.

12. The method of claim 1, further comprising communicating said pulse transit time to any of: a storage device, a display device, and a remote device over a network.

13. A system for determining pulse transit time for a subject, comprising:
a photodetector generating a time-series signal by sensing an intensity of light reflected off a region of exposed skin of a subject where a photoplethysmographic (PPG) signal can be detected, said time-series signal comprising said PPG signal;
a sensor placed on said subject's body where electrical activity of said subject's heart can be sensed, said sensor generating an electrocardiogram (ECG) signal; and
a processor and memory, said processor executing machine readable instructions for performing:
receiving said PPG and ECG signals; and
processing a temporally overlapping segment of said PPG and ECG signals to determine a pulse transit time between a reference point on said PPG signal and a reference point on said ECG signal.

14. The system of claim 13, wherein, in advance of processing said temporally overlapping segment of said PPG and ECG signals, further comprising compensating for motion in said PPG signal induced by a movement of said subject.

15. The system of claim 13, wherein, in advance of processing said temporally overlapping segment of said PPG and ECG signals, further comprising any of:
detrending said time-series signal to remove non-stationary components;
filtering said time-series signal with a cutoff frequency defined as a function of a frequency of said subject's cardiac pulse; and
performing automatic peak detection on said filtered signal.

16. The system of claim 13, wherein, in advance of processing said temporally overlapping segment of said PPG and ECG signals, further comprising temporally synchronizing said PPG and ECG signals.

17. The system of claim 13, wherein said reference point on said PPG signal is a characteristic point comprising any of: a maximum or a minimum point on said PPG signal, an average point between a maximum and a minimum on said PPG signal, a maximum of a first derivative of said PPG signal, and a maximum of a second derivative of said PPG signal.

18. The system of claim 13, wherein said reference point on said ECG signal is a peak point of a R wave.

19. The system of claim 13, wherein said reference point on said PPG signal and said reference point on said ECG signal are selected by a user.

20. The system of claim 13, wherein determining said pulse transit time (PTT) between said two reference points comprises:
calculating an instantaneous PTT at least comprising:
PTT=^{d}/_{η}_{_{HR}}
where η_{HR} is a frequency of said subject's cardiac pulse in beats/minute and d is a phase difference in radians/second between said two reference points on said temporally synchronous ECG and PPG signals; and
averaging said instantaneous PTT over at least two cardiac cycles.

21. The system of claim 13, wherein said PPG signal is extracted from said time-series signal.

22. The system of claim 13, further comprising determining, from said pulse transit time, any of: blood pressure, blood vessel dilation over time, blood vessel blockage, and blood flow velocity.

23. The system of claim 22, further comprising determining an occurrence of any of: cardiac arrhythmia, cardiac stress, heart disease, and peripheral vascular disease.

24. The system of claim 13, further comprising communicating said pulse transit time to any of: a storage device, a display device, and a remote device over a network.
